(19) United States
(12) Patent Application Publication
(10) Pub. No.: US 2022/0293221 A1
(43) Pub. Date: Sep. 15, 2022

(54) DATA STRUCTURE FOR GENOMIC INFORMATION

(71) Applicant: PREON VENTURES OY, Helsinki (FI)

(72) Inventor: Erkki HEILAKKA, Helsinki (FI)

(21) Appl. No.: 17/567,412

(22) Filed: Jan. 3, 2022

(30) Foreign Application Priority Data
Mar. 11, 2021 (FI) 20215266

(51) Int. Cl. G16B 50/30; G16B 50/50

(52) U.S. Cl. CPC G16B 50/30; G16B 50/50

(57) ABSTRACT

A method, comprising: obtaining data to be stored in a data structure comprising a first part and a second part, determining from the data a first data part that is to be stored in the first part of the data structure, wherein the first data part comprises a binary representation of a reference sequence and existence indicators for one or more variants, the said reference sequence comprising a plurality of base pairs, determining from the data a second data part that is to be stored in the second part of the data structure, wherein the second data part comprises a description of the said one or more variants, and storing the obtained data in the data structure such that the first data part is stored in the first data structure part and the second data part is stored in the second data structure part.

### FIELD

The present application relates to a data structure for storing genomic information.

### BACKGROUND

A genome is the complete set of genetic information in an organism. The study of the genome is called genomics. A genome comprises DNA, which may be understood as a code, and which varies from one individual to another. Knowing the variation of the genome of an individual may benefit in many ways, for example, in pharmacogenetics, which is a science of understanding how genetic variability influences drug response.

Genomic information may be traditionally stored in file formats that may require large memory or processing capacities. A data structure requiring less memory or processing capacities would bring the genomic information more easily and efficiently available to individuals and their caregivers.

### BRIEF DESCRIPTION

The scope of protection sought for various embodiments of the invention is set out by the independent claims. The exemplary embodiments and features, if any, described in this specification, that do not fall under the scope of the independent claims are to be interpreted as examples useful for understanding various embodiments of the invention.

According to another aspect there is provided an apparatus comprising means for obtaining data to be stored in a data structure comprising a first part and a second part, determining from the data a first data part that is to be stored in the first part of the data structure, wherein the first data part comprises a binary representation of a reference sequence and existence indicators for one or more variants, the said reference sequence comprising a plurality of base pairs, determining from the data a second data part that is to be stored in the second part of the data structure, wherein the second data part comprises a description of the said one or more variants, and storing the obtained data in the data structure such that the first data part is stored in the first data structure part and the second data part is stored in the second data structure part.

According to another aspect there is provided an apparatus comprising at least one processor, and at least one memory including a computer program code, wherein the at least one memory and the computer program code are configured, with the at least one processor, to cause the apparatus to obtain data to be stored in a data structure comprising a first part and a second part, determine from the data a first data part that is to be stored in the first part of the data structure, wherein the first data part comprises a binary representation of a reference sequence and existence indicators for one or more variants, the said reference sequence comprising a plurality of base pairs, determine from the data a second data part that is to be stored in the second part of the data structure, wherein the second data part comprises a description of the said one or more variants, and store the obtained data in the data structure such that the first data part is stored in the first data structure part and the second data part is stored in the second data structure part.

According to another aspect there is provided a method comprising obtaining data to be stored in a data structure comprising a first part and a second part, determining from the data a first data part that is to be stored in the first part of the data structure, wherein the first data part comprises a binary representation of a reference sequence and existence indicators for one or more variants, the said reference sequence comprising a plurality of base pairs, determining from the data a second data part that is to be stored in the second part of the data structure, wherein the second data part comprises a description of the said one or more variants, and storing the obtained data in the data structure such that the first data part is stored in the first data structure part and the second data part is stored in the second data structure part.

According to another aspect there is provided a computer program product readable by a computer and, when executed by the computer, configured to cause the computer to execute a computer process comprising obtaining data to be stored in a data structure comprising a first part and a second part, determining from the data a first data part that is to be stored in the first part of the data structure, wherein the first data part comprises a binary representation of a reference sequence and existence indicators for one or more variants, the said reference sequence comprising a plurality of base pairs, determining from the data a second data part that is to be stored in the second part of the data structure, wherein the second data part comprises a description of the said one or more variants, and storing the obtained data in the data structure such that the first data part is stored in the first data structure part and the second data part is stored in the second data structure part.

According to another aspect there is provided a computer program product comprising computer-readable medium bearing computer program code embodied therein for use with a computer, the computer program code comprising code for performing obtaining data to be stored in a data structure comprising a first part and a second part, determining from the data a first data part that is to be stored in the first part of the data structure, wherein the first data part comprises a binary representation of a reference sequence and existence indicators for one or more variants, the said reference sequence comprising a plurality of base pairs, determining from the data a second data part that is to be stored in the second part of the data structure, wherein the second data part comprises a description of the said one or more variants, and storing the obtained data in the data structure such that the first data part is stored in the first data structure part and the second data part is stored in the second data structure part.

According to another aspect there is provided a computer program comprising instructions for causing an apparatus to perform at least the following: obtaining data to be stored in a data structure comprising a first part and a second part, determining from the data a first data part that is to be stored in the first part of the data structure, wherein the first data part comprises a binary representation of a reference sequence and existence indicators for one or more variants, the said reference sequence comprising a plurality of base pairs, determining from the data a second data part that is to be stored in the second part of the data structure, wherein the second data part comprises a description of the said one or more variants, and storing the obtained data in the data structure such that the first data part is stored in the first data structure part and the second data part is stored in the second data structure part.

According to another aspect there is provided a computer readable medium comprising program instructions for causing an apparatus to perform at least the following: obtaining data to be stored in a data structure comprising a first part and a second part, determining from the data a first data part that is to be stored in the first part of the data structure, wherein the first data part comprises a binary representation of a reference sequence and existence indicators for one or more variants, the said reference sequence comprising a plurality of base pairs, determining from the data a second data part that is to be stored in the second part of the data structure, wherein the second data part comprises a description of the said one or more variants, and storing the obtained data in the data structure such that the first data part is stored in the first data structure part and the second data part is stored in the second data structure part.

According to another aspect there is provided a non-transitory computer readable medium comprising program instructions for causing an apparatus to perform at least the following: obtaining data to be stored in a data structure comprising a first part and a second part, determining from the data a first data part that is to be stored in the first part of the data structure, wherein the first data part comprises a binary representation of a reference sequence and existence indicators for one or more variants, the said reference sequence comprising a plurality of base pairs, determining from the data a second data part that is to be stored in the second part of the data structure, wherein the second data part comprises a description of the said one or more variants, and storing the obtained data in the data structure such that the first data part is stored in the first data structure part and the second data part is stored in the second data structure part.

### BRIEF DESCRIPTION OF THE DRAWINGS

FIG. 1 illustrates an exemplary embodiment of an environment storing or processing genetic information.

FIG. 2 illustrates an exemplary embodiment of a data structure.

FIG. 3 illustrates an exemplary embodiment of a data structure comprising genomic data.

FIG. 4 illustrates a flow chart according to an exemplary embodiment.

FIG. 5 illustrates an exemplary embodiment of a part of a data structure.

FIG. 6 illustrates an exemplary embodiment of an apparatus.

### DETAILED DESCRIPTION

DNA sequencing may be understood as a process of determining the nucleic acid sequence, that is, the order of nucleotides in a genome. Nucleotides may be composed of three subunit molecules: a nucleobase, a five-carbon sugar, and a phosphate. The nucleobases in DNA are adenine (A), guanine (G), cytosine (C), and thymine (T), which form pairs as follows: adenine with thymine and guanine with cytosine. Nucleobases that are bonded together as pairs may be called base pairs. A base pair may be unambiguously presented by the letter of the first nucleobase of the pair. DNA sequencing comprises a method or a technology that may be utilized to determine the order of the nucleobases in a genome.

In next-generation sequencing (NGS), also known as high-throughput sequencing, a genome is usually broken into blocks of DNA, which may be called reads. These reads may comprise 100-300 base pairs or up to 2 million base pairs depending on the utilized sequencing device. In the sequencing process the base pairs of a read may be associated with quality scores.

Due to a high probability of sequencing errors, a genome is sequenced multiple times. The number of overlapping reads, that is, the number of unique reads comprising a given position in the multiple sequences may be called depth, or sequencing depth. For example, a sequencing depth of 30 may be understood as that the nucleotides in a genome are sequenced on average 30 times. The depth is not uniform, and therefore some parts of a sequenced genome may have a small number of overlapping reads, for example 5, and some parts may have a large number of overlapping reads, for example 100. A set of reads covering a position may establish a pileup. The number of reads in a pileup may be the same as the sequencing depth at the position of the pileup.

Reads may not have mapping information associated with them. The positions of reads in a genome may be determined by using an alignment algorithm. An alignment algorithm compares reads to a reference sequence that has been assembled as a representative example of the genome of an idealised individual organism of a species. An alignment algorithm may be utilized to obtain aligned reads, which may be understood as reads that have been positioned in relation to a reference sequence. FASTQ format is an example of how unaligned reads may be stored. In FASTQ format a text-based format is used for storing an output of high-throughput sequencing instruments, that is, unaligned reads, and corresponding quality scores.

A reference genome may be represented as FASTA format. In FASTA format a text-based format is used for representing nucleotide sequences or amino acid sequences. Another format for storing DNA sequences is 0.2 bit file format. In 0.2 bit file format multiple DNA sequences may be stored in a randomly accessible format. The 0.2 bit file format comprises various 32-bit fields and some fields of other sizes. The fields may comprise information of the sequence, for example, sequence name, size, and masking information of the sequence. A masked sequence may be understood as a sequence, where repetitive or low-complexity regions of the sequence have been replaced with “N” in order not to be detected by an analysis algorithm. A DNA sequence is stored in 0.2 bit file format in a field called packedDNA using two bits per base pair.

There are multiple file formats for storing aligned reads. SAM (Sequence Alignment/Map) and its compressed binary version BAM are examples of formats that could be used. CRAM is another format that may be used to achieve a higher compression ratio. MPEG-G format may be more versatile since it may be used for replacing multiple traditional formats in genomic processing.

An individual's genomic data may be understood as an individual's genomic sequence that may be compared to a reference sequence. An individual's genomic sequence differs from the corresponding reference sequence. Such differences may be called variants. A variant may be a single-nucleotide polymorphism (SNP), which is a substitution of a single nucleotide at a specific position in the genome. A variant may also be a structural divergence covering multiple nucleotides, for example, an insertion or a deletion of a sequence (InDel). An average difference between the genomes of two individuals may be estimated as 20 million base pairs, or 0.6% of the total of 3.2 billion base pairs.

Variants may be identified by using a variant calling algorithm. Such algorithms comprise, for example, prior genotype probability estimation, error models for data observations, partitioning of the genotype, heuristic based algorithms, and machine learning based techniques. A variant calling algorithm may compare, position by position, a pileup of reads, which may be understood as a set of reads covering a position, to the reference sequence. Identified variants, that is, called variants may be stored in VCF (Variant Calling Format) file format or its binary version BCF file format. A line in the body of a VCF file represents a variant. Another file format that may be used for storing variants is GVF (Genomic Variation Format).

Variant annotation may be understood as a process of assigning information to DNA and determining the impact and significance of called variants. A variant annotation algorithm may compare called variants with variant databases. Characteristics of previously unknown variants may be predicted using in-silico algorithms. The outcome of a variant annotation process may be a human-readable report in PDF or a similar sharable file format.

The file formats presented above are examples of file formats designed for supporting next-generation sequencing (NGS) genomic analysis pipelines. They may be processed in servers with large memory capacities and powerful CPUs. Various solutions have been developed to store extensive files. When processing, comparing, or visualising a genome in a user device with limited processing and memory capacities, the use of these file formats may be inefficient or impossible.

A combination of a reference genome and called variants, that is, the variants identified by a variant calling algorithm, may be sufficient for reconstructing a genome of an individual, and therefore it may provide a basis for analysis. Also aligned reads may be stored for enabling human experts visually verify the quality of the alignment and/or the variant calling results.

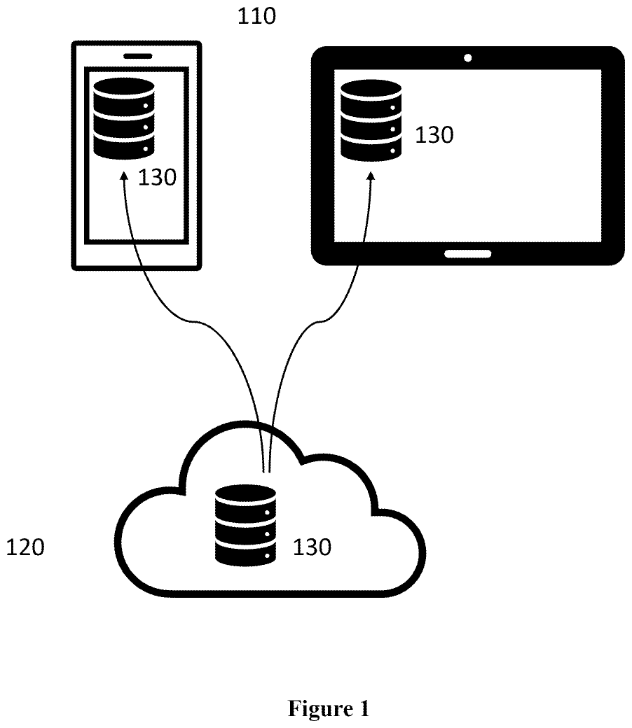

FIG. 1 presents an exemplary embodiment of an environment suitable for storing or processing genetic information, which would benefit from a data structure that does not need a large memory or processing capacity. In this context an environment may be understood as a set-up comprising one or more user devices, one or more databases, and/or one or more genomic datasets, or a combination thereof. A user device 110 may be for example a mobile phone, a tablet, or any other device capable of connecting to the internet and optionally also installing applications. A cloud 120 may be a computational unit comprising one or more computing devices, a database, a computer unit, or a combination thereof, comprising storage capacity and capable of doing cloud computing. The cloud 120 comprises genomic data 130 and may send the genomic data 130 to the user device 110. The genomic data 130 may be encoded in the cloud 120 to a format suitable for the user device 110, the cloud 120 may obtain the data from another device or server in a format suitable for the user device 110, or the data may be encoded to a suitable format in the user device 110.

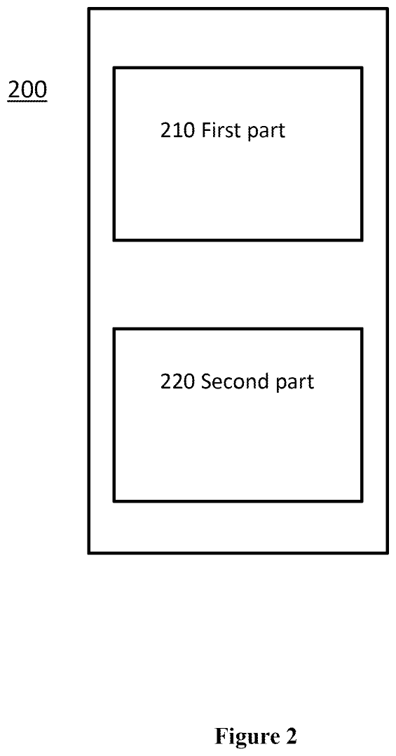

FIG. 2 illustrates an exemplary embodiment of a data structure 200 that would facilitate storing, processing, or utilizing genomic data on a user device 110 that may have limited processing and memory capacities. Obtaining data to be stored in the data structure 200 may be understood as using methods described above to obtain data from an individual's genomic data, or it may be understood as obtaining data that is in a suitable format for the data structure 200 from another device or cloud. Obtaining data to be stored in the data structure 200 may comprise encoding data to a format suitable for the data structure 200. Obtaining data to be stored in the data structure 200 may comprise formatting a reference sequence to a binary representation and adding variance existence indicators to the binary representation, or it may comprise obtaining a binary representation from another device or cloud. Variance existence indicator may be understood as data that expresses existence or absence of a variant. Obtaining data to be stored in the data structure 200 may comprise forming variant descriptions of called variants in a textual, binary, or other format, or it may comprise obtaining variant descriptions of called variants from another device or cloud. The data structure 200 comprises a first part 210 and a second part 220. The first part of the data structure 210 may be used for storing a first part of an individual's genomic data as a first data part and the second part of the data structure 220 may be used for storing a second part of the same individual's genomic data as a second data part. The data may be organized such that the first data part comprises information of in which nucleotides the individual's genome differs from a reference genome, and the second data part comprises information of how the individual's genome differs from the reference genome in the nucleotides indicated in the first data part. The first data part may be determined from the obtained data by extracting the binary representation described above from the obtained data, or it may be determined from the obtained data by using methods described above from the individual's genomic data by formatting the reference sequence to a binary representation and adding existence indicators for variants to the binary representation. The second data part may be determined from the obtained data by extracting the variant descriptions of called variants described above from the obtained data, or it may be determined from the obtained data by obtaining it using methods described above from the individual's genomic data by forming variant descriptions of called variants in a textual, binary, or other format. This partition to a first data part and a second data part may enable the genomic information to be stored in an effective and low-memory data structure, and the data structure may be stored, for example, to a user device with limited processing and memory capacities. This may enable an individual to possess and use their private genetic information without having to transfer private data over an Internet access. The data structure 200 differs from the 0.2 bit file format by providing variant existence and variant description information in addition to the reference genome in a compact format that enables storing the information in a user device with limited processing and memory capacities, for example, a mobile device. Variant information enables the genomic data to be individualized and therefore used, for example, for personalized medicine in pharmacogenomics. Pharmacogenomics may be understood as a study of how genomics affects personal physiological response to medication. The data structure 200 may enable an individual to bring their private genetic information with them in a user device to, for example, a doctor's office or a hospital to be available for example for personalized medication planning to prevent adverse drug reactions or estimate treatment response.

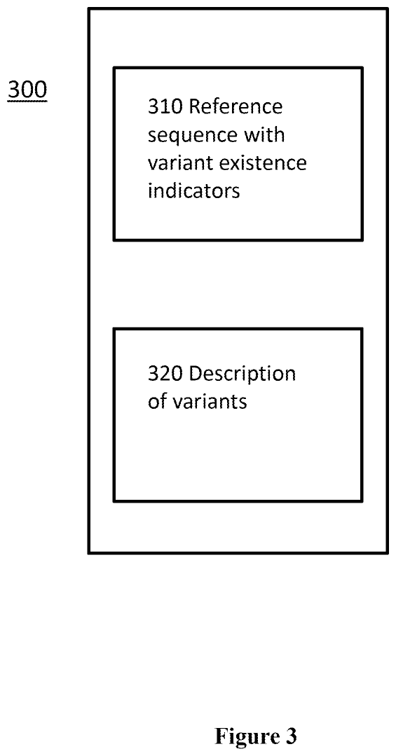

FIG. 3 illustrates an exemplary embodiment of a data structure such as presented in FIG. 2 comprising genomic data 300. A first data part 310 is stored in the first part of the data structure 210. The first data part 310 comprises a binary representation of a reference sequence and variance existence indicators at positions of the reference sequence that correspond to existing variants. The reference sequence is organized as nucleotides of a chromosome. The first data part 310 comprises the content of nucleotides and comprises also the information whether or not the individual's genome differs from the reference sequence at the positions of the nucleotides, respectively. The first data part 310 may further comprise additional information of the said chromosome. The additional information may be organized at the beginning of the first data part 310. For example, a first byte of the first data part 310 may comprise the number of the said chromosome, and following bytes of the first data part 310 may further comprise additional information of the sequence, for example, length, first position, last position, or other information. Additionally, or alternatively, additional information of the said chromosome may be organized differently in the first data part 310. A second data part 320 is stored in the second part of the data structure 220. The second data part 320 comprises a description of variants. Alternatively, or additionally, the second data part 320 may comprise one or more links that point to data files comprising variant description information. The data structure 200 may be used, for example, by first reviewing the first data part 310 for information of variation existence in the individual's genome. If variation in an interesting or a significant nucleotide is detected, the second data part 320 may be reviewed for further studying the characteristics of the variation. The first data part 310 and/or the second data part 320 may be encrypted entirely or partly to further increase privacy.

Additionally, the data structure 200 may comprise headers. A header may comprise supplemental information about the data structure 200, for example information of the number of bytes or bits used for storing data in the data structure 200, in the first part of the data structure 210, and/or the second part of the data structure 220. Additionally, or alternatively, a header may comprise semantic information of the data or part of the data. The data structure 200 may comprise a header, wherein the header is part of the data structure 200. Additionally, or alternatively, the data structure 200 may comprise a first data part header that is comprised in the first data part 310 and/or a second data part header that is comprised in the second data part 320.

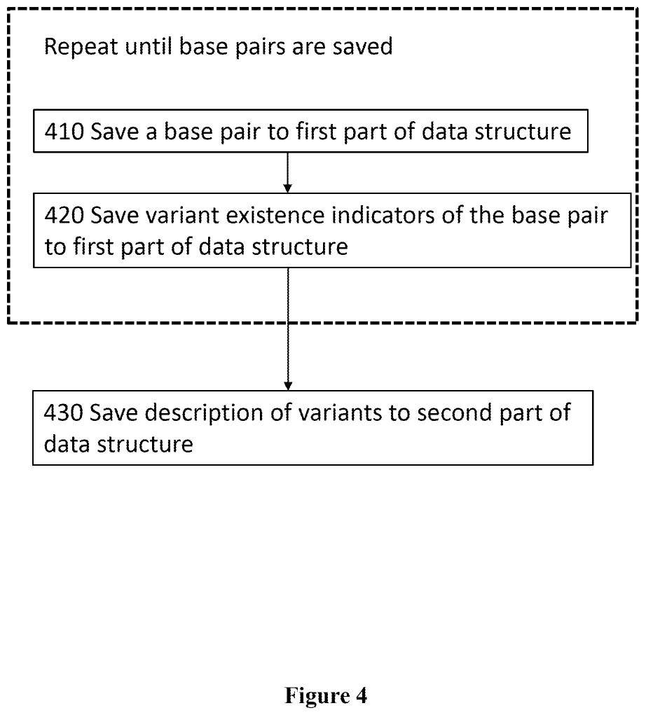

FIG. 4 illustrates a flow chart according to an exemplary embodiment of storing genomic data to a two-part data structure 300. For example, there may be an individual, whose genome has been sequenced by DNA sequencing on the basis of a reference sequence. The individual may benefit from possessing that private genomic information on a user device, where the data may be secure and easily accessible by the owner. Therefore, the data may be saved in such a data structure that the user device may contain and process. The reference sequence may be saved in the first part of the data structure 210. This may be executed one base pair at a time. A base pair of a reference sequence is saved 410 to the first part of the data structure 210. A variance existence indicator corresponding to said base pair is saved 420 to the first part of the data structure 210. Steps 410 and 420 may be repeated for saving a plurality of base pairs until the reference sequence is saved in the first part of the data structure 210. The data saved in the first part of the data structure 210 forms the first data part 310. Descriptions of the variants are saved 430 to the second part of the data structure 220 and they form the second data part 320. Alternatively, step 430 may be executed before steps 410 and 420, and/or it may be executed recurrently whenever a base pair is saved 410 to the first part of the data structure 210 and the said base pair has a variant for which the description is not yet saved in the second part of the data structure 220. This data saving to the individual's user device may be executed by an operator that has performed the DNA sequencing, or additionally, or alternatively by a third party. Genomic data may be saved to a user device also partially and may be complemented later, for example comprising a reference sequence at first and complemented later with variance existence information, or comprising a partially sequenced data at first and complemented with more individual variant information later. The first data part 310 is aligned with base pairs and in this exemplary embodiment adjacent bytes have no semantic connection to each other. Therefore, the first data part 310 may be processed and compared efficiently by parallel processing, for example by graphics processing units (GPUs).

In one exemplary embodiment of the data structure 200, one base pair of a reference sequence with variance existence indicators may be stored using eight bits, that is, one byte. A first bit may indicate the existence or absence of a value in the reference genome, for example with 1 for existence and 0 for absence. A second bit and a third bit may represent four possible base pair values (A, G, C, T), for example with 00 for A, 01 for G, 10 for C, and 11 for T. A fourth bit may indicate the existence or absence of a value in the individual's genome, for example with 1 for existence and 0 for absence. A fifth bit may indicate the existence or absence of a variant, for example with 1 for existence and 0 for absence. A sixth bit, a seventh bit, and an eighth bit may be reserved for other information. The values stored in the said eight bits may also be arranged in a different order.

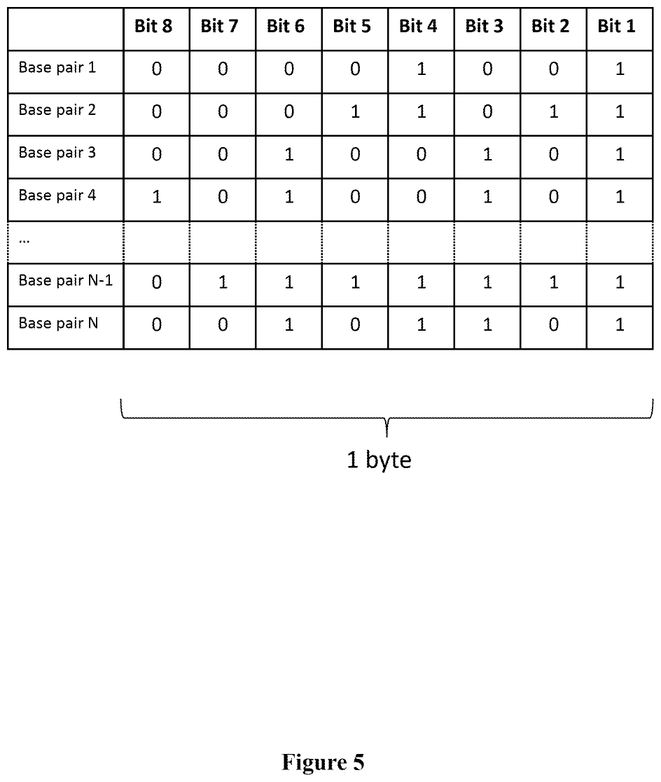

FIG. 5 illustrates an exemplary embodiment of the first part of the data structure 210 and the first data part 310 stored in the first part of the data structure 210. Data concerning one base pair may be stored using eight bits. In this exemplary embodiment base pair 1 has a value in the reference genome, and therefore the value 1 has been stored in bit 1 of the byte that is represented on the row indicated by Base pair 1 in FIG. 5. Further, the first nucleotide of base pair 1 is adenine (A), and therefore values 0 and 0 have been stored in bits 2 and 3. The individual's genome does have a value in base pair 1, and therefore the value 1 has been stored in bit 1. The individual's genome does not have a variant in base pair 1, and therefore the value 0 has been stored in bit 5. The values stored in bits 6, 7 and 8 may indicate other information concerning the base pair. Other base pairs have their own bytes in the first part of data structure 210 and data concerning these base pairs of the first data part 310 have been stored in the bits of these bytes, respectively.

In one exemplary embodiment of the data structure 200, two or three of the reserved bits may indicate a relative depth at the position of the base pair. A maximum depth may be understood as the number of overlapping reads in the highest pileup in the sequenced DNA. A maximum depth may also be understood as a pre-defined numerical value that may be stored in the data structure 200. The pre-defined numerical value may be determined, for example, on the basis of the number of overlapping reads of the pileups in the sequenced DNA, on the basis of the number of overlapping reads of pileups in some previously sequenced DNAs, or by some other means. A relative depth may be understood as the number of overlapping reads at a certain position of the DNA expressed as a percentage of the maximum depth. Alternatively, the relative depth could be calculated, for example, in relation to the mean depth, average depth, or a pre-defined threshold depth. For example, in binary data 00 may mean a depth of less than or equal to 25% of the maximum depth, 01 a depth of more than 25% but less than or equal to 50% of the maximum depth, 10 a depth of more than 50% but less than or equal to 75% of the maximum depth, and 11 a depth of more than 75% but less than or equal to 100% of the maximum depth.

In one exemplary embodiment of the data structure 200, one of the reserved bits may indicate an existence of extra information corresponding to the base pair of the reference sequence indicated in the first, second and third bits of the eight bits, which may be stored in a separate part of the data structure. For example, 1 may indicate that extra information exists corresponding to the said base pair and 0 may indicate that no extra information exists corresponding to the said base pair. The extra information could be, for example, variant calling results, quality measures, or other information. In one exemplary embodiment of the data structure 200, one of the reserved bits may indicate if a variant in that position is a single nucleotide polymorphism (SNP) or insertion and deletion (InDel).

In one exemplary embodiment of the data structure 200, one of the reserved bits may indicate if a variant in that position is heterozygous or not. Heterozygous may be understood as having inherited different forms of a particular gene from each parent, as opposed to homozygous, in which identical forms of a particular gene have been inherited from both parents.

In one exemplary embodiment of the data structure 200, one of the reserved bits may indicate an acceptable quality of the pileup at the corresponding position, for example 0 indicating that the quality is not acceptable and 1 indicating that the quality is acceptable. The quality of the pileup could be related to the number of reads at the corresponding position, or alternatively to some other quality measure of the sequencing at the corresponding position. An acceptable quality may be understood as a quality measure value that exceeds a threshold that has been set as an acceptability threshold. The acceptability threshold of quality may be determined, for example, in relation to an average quality of the sequence, in relation to a maximum quality measure of the sequence, or by some other means.

In one exemplary embodiment of the data structure 200, one base pair of a reference sequence with variance existence indicators may be stored using four bits. Therefore, one byte in the data structure 200 may comprise two base pairs instead of one base pair, which would half the storage size needed for the reference sequence in comparison to an exemplary embodiment that would store one base pair using one byte, that is, eight bits. This reduction in storage capacity requirement would further bring the genomic information more easily and efficiently available. In an exemplary embodiment of four bits per one base pair a first bit may indicate the existence or absence of a value in the reference genome, for example with 1 for existence and 0 for absence. A second bit and a third bit may represent four possible base pair values (A, G, C, T), for example with 00 for A, 01 for G, 10 for C, and 11 for T. A fourth bit may indicate the existence or absence of a variant, for example with 1 for existence and 0 for absence. An absence of a value in the individual's genome may be indicated as an existence of a variant and described in the second data part 320 of the data structure 200. The values stored in the said four bits may also be arranged in a different order.

In one exemplary embodiment of the data structure 200, one base pair of a reference sequence with variance existing indicators may be stored using three bits. A first bit and a second bit may represent four possible base pair values (A, G, C, T), for example with 00 for A, 01 for G, 10 for C, and 11 for T, and a third bit may indicate the existence or absence of a variant, for example with 1 for existence and 0 for absence. The values stored in the said three bits may also be arranged in a different order.

In one exemplary embodiment of the data structure, one base pair of a reference sequence with variance existing indicators may be stored using four bits. A first bit and a second bit may represent four possible base pair values (A, G, C, T), for example with 00 for A, 01 for G, 10 for C, and 11 for T, and a third bit may indicate the existence or absence of a variant, for example with 1 for existence and 0 for absence. A fourth bit may indicate the existence or absence of a value in the reference genome, for example with 1 for existence and 0 for absence. The values stored in the four bits may also be arranged in a different order.

In one exemplary embodiment of the data structure, one base pair of a reference sequence with variance existing indicators may be stored using five bits. A first bit and a second bit may represent four possible base pair values (A, G, C, T), for example with 00 for A, 01 for G, 10 for C, and 11 for T, and a third bit may indicate the existence or absence of a variant, for example with 1 for existence and 0 for absence. A fourth bit may indicate the existence or absence of a value in the reference genome, for example with 1 for existence and 0 for absence, and a fifth bit may indicate the existence or absence of a value in the individual's genome, for example with 1 for existence and 0 for absence. The values stored in the five bits may also be arranged in a different order.

In one exemplary embodiment of the data structure, one base pair of a reference sequence with variance existing indicators may be stored using six, seven or eight bits. A first bit and a second bit may represent four possible base pair values (A, G, C, T), for example with 00 for A, 01 for G, 10 for C, and 11 for T, and a third bit may indicate the existence or absence of a variant, for example with 1 for existence and 0 for absence. A fourth bit may indicate the existence or absence of a value in the reference genome, for example with 1 for existence and 0 for absence, and a fifth bit may indicate the existence or absence of a value in the individual's genome, for example with 1 for existence and 0 for absence. A sixth bit, a seventh bit and an eighth bit may indicate other information. This other information may comprise indicating a relative depth at the position of each base pair, an acceptable quality of a pile-up at the position of each base pair, an existence of a single nucleotide polymorphism or insertion and deletion as a variant in the position of each base pair, an existence of a heterozygous or homozygous variant in the position of each base pair, and/or an existence of extra information for each base pair, wherein the said extra information is stored in the second part of the data structure. The values stored in the eight bits may also be arranged in a different order.

In one exemplary embodiment of the data structure 200, variant calling results may be stored as a separate block in the data structure for example as a VCF, VGF or a similarly formatted field.

In one exemplary embodiment of the data structure 200 the second data part 320 may comprise a plurality of indexes pointing to chromosomes and/or variant description blocks. Alternatively, the said plurality of indexes may be comprised in a header that is part of the data structure 200, in a first data part header that is comprised the first data part 310, and/or in a second data part header that is comprised in the second data part 320.

In one exemplary embodiment of the data structure 200, variant calling results may comprise results of multiple variant calling algorithms, and the variant calling results may be grouped according to the algorithms or tagged with an algorithm identifier.

In one exemplary embodiment of the data structure 200, variant calling results may be stored on an external server and/or in an internal memory of a user device, and the data structure may comprise IDs or URLs for identifying the variant calling result data on the external server and/or in the internal memory of the user device.

In one exemplary embodiment of the data structure 200, in addition to variant calling results, variant description information may be stored in the second part of the data structure 220 as a textual data or as a binary representation. Variant description information may comprise, for example, pileups of unaligned reads at the position of a variant, quality scores, and/or aligned reads.

In one exemplary embodiment of the data structure 200, aligned reads may be stored as a separate block in the data structure. This would enable visual verification of the quality of the alignment and/or the variant calling results.

In one exemplary embodiment of the data structure 200, the data structure or a part of the data structure may be compressed by using a data compression method.

In one exemplary embodiment of the data structure 200, the data structure may be stored in a file.

In one exemplary embodiment of the data structure 200, the data structure may be stored in a memory of a user device 110.

The exemplary embodiments described above may also be combined, which would be clear to a skilled person. For example, the features introduced in the exemplary embodiments, or a combination of some of the features, may be present simultaneously in some other exemplary embodiments.

The data structure 200 may facilitate accessing the genomic information. A byte-aligned representation of a reference genome may enable an application to read a single base pair or a set of base pairs using direct access to the byte array. Assuming that the position of the first base pair is 0, the position of a base pair is the index in the byte array.

The data structure 200 facilitates comparing two or more genomes. Similarities and differences of the two or more genomes may be found with a bit-level comparison. In the data structure 200 a reference sequence may be stored together with variance existence indicators. This may enable visualizing or processing a large number of base pairs, for example a whole chromosome, without requiring a significant amount of time, memory, or processing power. Additionally, the data may be visualized with quality and/or depth indicators.

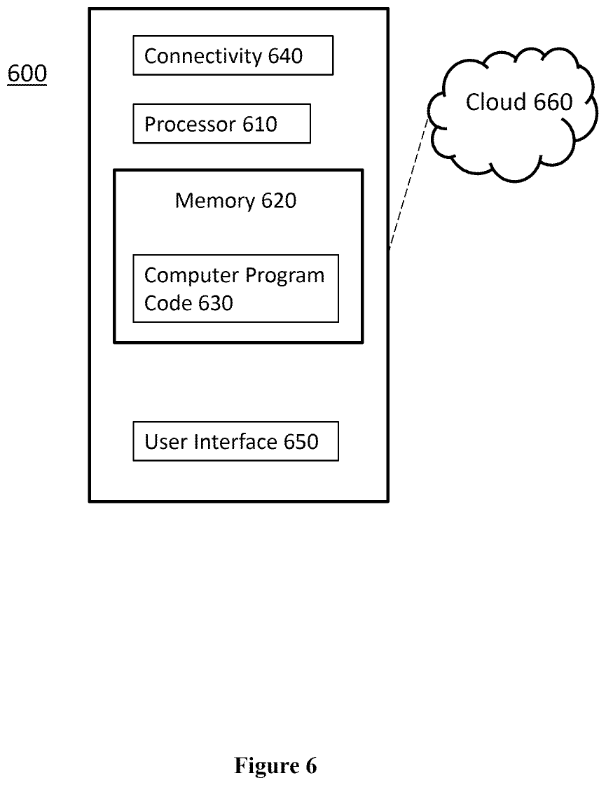

FIG. 6 illustrates an exemplary embodiment of an apparatus such as user device 110. The apparatus is applicable for performing one or more exemplary embodiments of the invention. The apparatus 600 comprises a processor 610 that may comprise one or more processing cores containing circuitry configured to executed instructions comprised in computer program code 630. The computer program code 630 may be any such computer program code which can be stored, temporarily or permanently, in the memory 620 and executed by the processor 610. The memory 620 may be implemented using any suitable data storage technology, such as semiconductor-based memory devices, flash memory, magnetic memory devices and systems, optical memory devices and systems, fixed memory, and removable memory. The apparatus 600 may further comprise a connectivity unit 640 configured to enable wired and/or wireless connectivity. The wireless connectivity may be cellular connectivity, Wi-Fi connectivity, Bluetooth connectivity, and/or any other suitable method of connectivity. A user interface 650 enables a user to interact with the apparatus 600. The user interface 650 may comprise for example a display, a loudspeaker, a keyboard, a mouse, a touch user interface, and/or a microphone. Any other suitable means for user interaction could also be comprised in the user interface 650.

1. A method, comprising:
obtaining data to be stored in a data structure comprising a first part and a second part;
determining from the data a first data part that is to be stored in the first part of the data structure, wherein the first data part comprises a binary representation of a reference sequence and existence indicators for one or more variants, the said reference sequence comprising a plurality of base pairs;
determining from the data a second data part that is to be stored in the second part of the data structure, wherein the second data part comprises a description of the said one or more variants; and
storing the obtained data in the data structure such that the first data part is stored in the first data structure part and the second data part is stored in the second data structure part.

2. A method according to claim 1, wherein the data structure further comprises:
a header, wherein the header is part of the data structure;
a first data part header that is comprised in the first data part; and/or
a second data part header that is comprised in the second data part.

3. A method according to claim 1, wherein the plurality of base pairs of the said first data part is stored using two bits for each base pair value and using one bit for a variant existence indicator for each base pair.

4. A method according to claim 3, wherein the said plurality of base pairs is stored using one bit for a reference genome value indicator for each base pair.

5. A method according to claim 4, wherein the said plurality of base pairs is stored using one bit for a value indicator in an individual's genome for each base pair.

6. A method according to claim 5, wherein the said plurality of base pairs is stored using one or more bits to indicate one or more of the following:
a relative depth at the position of each base pair;
an acceptable quality of a pile-up at the position of each base pair;
an existence of a single nucleotide polymorphism or insertion and deletion as a variant in the position of each base pair;
an existence of a heterozygous or homozygous variant in the position of each base pair; and/or
an existence of extra information for each base pair, wherein the said extra information is stored in the second part of the data structure.

7. A method according to claim 1, wherein the second data part comprises a plurality of indexes that point to chromosomes and variant description blocks.

8. A method according to claim 2, wherein the said header, the said first data part header, and/or the said second data part header comprises a plurality of indexes that point to chromosomes and variant description blocks.

9. A method according to claim 1, wherein the said second data part comprises variant calling result data from one or more variant calling algorithms.

10. A method according to claim 9, wherein the said variant calling result data are grouped according to the said one or more variant calling algorithms and/or tagged with a variant calling algorithm identifier.

11. A method according to claim 9, wherein the said second data part comprises variant description information.

12. A method according to claim 1, wherein the second data part comprises one or more identifiers and/or Uniform Resource Locators that point to an internal memory of a user device and/or an external server comprising variant calling result data.

13. A method according to claim 1, wherein the method further comprises one or more of the following:
the data or part of the data in the data structure is compressed using a data compression method;
the data or part of the data in the data structure is encrypted entirely or partly;
and/or the data structure is stored in a file or in a memory of a user device.

14. An apparatus comprising at least one processor, and at least one memory including a computer program code, wherein the at least one memory and the computer program code are configured, with the at least one processor, to cause the apparatus to:
obtain data to be stored in a data structure comprising a first part and a second part;
determine from the data a first data part that is to be stored in the first part of the data structure, wherein the first data part comprises a binary representation of a reference sequence and existence indicators for one or more variants, the said reference sequence comprising a plurality of base pairs;
determine from the data a second data part that is to be stored in the second part of the data structure, wherein the second data part comprises a description of the said one or more variants; and
store the obtained data in the data structure such that the first data part is stored in the first data structure part and the second data part is stored in the second data structure part.

15. An apparatus according to claim 14, wherein the data structure further comprises:
a header, wherein the header is part of the data structure;
a first data part header that is comprised in the first data part; and/or
a second data part header that is comprised in the second data part.

16. An apparatus according to claim 14, wherein the plurality of base pairs of the said first data part is stored using two bits for each base pair value and using one bit for a variant existence indicator for each base pair.

17. An apparatus according to claim 16, wherein the said plurality of base pairs is stored using one bit for a reference genome value indicator for each base pair.

18. An apparatus according to claim 17, wherein the said plurality of base pairs is stored using one bit for a value indicator in an individual's genome for each base pair.

19. An apparatus according to claim 14, wherein the second data part comprises a plurality of indexes that point to chromosomes and variant description blocks

20. A non-transitory computer readable medium comprising program instructions for causing an apparatus to perform at least the following:
obtaining data to be stored in a data structure comprising a first part and a second part;
determining from the data a first data part that is to be stored in the first part of the data structure, wherein the first data part comprises a binary representation of a reference sequence and existence indicators for one or more variants, the said reference sequence comprising a plurality of base pairs;
determining from the data a second data part that is to be stored in the second part of the data structure, wherein the second data part comprises a description of the said one or more variants; and
storing the obtained data in the data structure such that the first data part is stored in the first data structure part and the second data part is stored in the second data structure part.
